(19) United States
(12) Patent Application Publication
(10) Pub. No.: US 2023/0200824 A1
(43) Pub. Date: Jun. 29, 2023

(54) MEDICAL CLIP AND METHOD FOR PRODUCING A MEDICAL CLIP

(71) Applicant: Aesculap AG, Tuttlingen (DE)

(72) Inventors: Thomas Pleil, Bad Duerrheim (DE); Thomas Scholten, Tuttlingen (DE); Michael Willmann, Villingen-Schwenningen (DE); Alexander Happle, Huefingen (DE); Jens Beger, Tuttlingen (DE)

(21) Appl. No.: 18/113,766

(22) Filed: Feb. 24, 2023

Related U.S. Application Data
(63) Continuation of application No. PCT/EP2021/073763, filed on Aug. 27, 2021.

(30) Foreign Application Priority Data
Aug. 27, 2020 (DE) 10 2020 122 392.7

(51) Int. Cl. A61B 17/122

(52) U.S. Cl. CPC A61B 17/1227; A61B 2017/00526

(57) ABSTRACT

A medical clip includes first and second clamping arms and a biasing element. The first clamping arm is connected to a first end of the biasing element, and the second clamping arm is connected to a second end of the biasing element. The first clamping arm and the second clamping arm are maximally proximate to one another in a basic position of the clip and are movable away from one another against the action of the biasing element from the basic position into an opening position. The medical clip is made partially by a generative manufacturing process, in which the medical clip has at least one first clip portion and at least one second clip portion. The at least one second clip portion is formed directly onto the at least one first clip portion by a generative manufacturing process.

### CROSS-REFERENCE TO RELATED APPLICATIONS

This application is a continuation of International Application No. PCT/EP2021/073763, filed on Aug. 27, 2021, and claims priority to German Application No. 10 2020 122 392.7, filed on Aug. 27, 2020. The contents of International Application No. PCT/EP2021/073763 and German Application No. 10 2020 122 392.7 are incorporated by reference herein in their entireties.

### FIELD

The present invention relates to medical clips generally, and more specifically to a medical clip, in particular in the form of an aneurysm clip, which comprises a first clamping arm, a second clamping arm, and a biasing element, wherein the first clamping arm is connected to a first end of the biasing element, wherein the second clamping arm is connected to a second end of the biasing element, and wherein the first clamping arm and the second clamping arm are maximally proximate to one another, in particular abutting against one another, in a basic position of the clip, and are movable away from one another against the action of the biasing element from the basic position into an opening position.

Furthermore, the present invention relates methods for producing medical clips generally, and more specifically to a method for producing a medical clip, in particular in the form of an aneurysm clip, wherein the clip is configured having a first clamping arm, a second clamping arm, and a biasing element such that the first clamping arm is connected to a first end of the biasing element, that the second clamping arm is connected to a second end of the biasing element, and that the first clamping arm and the second clamping arm are maximally proximate to one another, in particular abutting against one another, in a basic position of the clip, and are movable away from one another against the action of the biasing element from the basic position into an opening position.

### BACKGROUND

Medical clips of the kind described at the outset are known in a wide range of embodiments. Examples are described, in particular, in DE 10 2018 103 903 A1.

Clips of the kind described at the outset are used, in particular, in the form of aneurysm clips for the neurosurgical treatment of cranial aneurysms. During the treatment, it is important that the clip that fits the shape and size of the aneurysm is applied. Therefore, in particular, neurosurgeons require a large number of different aneurysm clips that differ from one another in their size, in the shape of the clamping arms, also referred to as the mouth part geometry, and in the closing force. In practice, this leads, in particular, to a surgeon selecting the respective best suited clip from several hundred different medical clips for an optimal treatment of an aneurysm, in order to clamp off an aneurysm.

The production of such aneurysm clips requires numerous process steps. This leads to a tool-intensive and expensive production of the clips.

### SUMMARY

In a first aspect of the invention, a medical clip is provided, in particular in the form of an aneurysm clip. The medical clip comprises a first clamping arm, a second clamping arm, and a biasing element. The first clamping arm is connected to a first end of the biasing element. The second clamping arm is connected to a second end of the biasing element. The first clamping arm and the second clamping arm are maximally proximate to one another, in particular abutting against one another, in a basic position of the clip and are movable away from one another against the action of the biasing element from the basic position into an opening position. The medical clip is made at least partially, in particular completely, by a generative manufacturing process. The medical clip comprises at least one first clip portion and at least one second clip portion. The at least one second clip portion is formed directly onto the at least one first clip portion by a generative manufacturing process.

In a second aspect of the invention, a medical clip is provided, in particular in the form of an aneurysm clip. The medical clip comprises a first clamping arm, a second clamping arm, and a biasing element. The first clamping arm is connected to a first end of the biasing element. The second clamping arm is connected to a second end of the biasing element. The first clamping arm and the second clamping arm are maximally proximate to one another, in particular abutting against one another, in a basic position of the clip and are movable away from one another against the action of the biasing element from the basic position into an opening position. The biasing element is configured in the form of a coil spring with at least one complete winding. A winding receptacle is formed on the biasing element in the region of the first end and/or the second end. The winding receptacle is formed pointing in the direction toward the adjacent winding.

In a third aspect of the invention, a method for producing a medical clip, in particular in the form of an aneurysm clip, is proposed. The clip is configured having a first clamping arm, a second clamping arm, and a biasing element, such that the first clamping arm is connected to a first end of the biasing element, that the second clamping arm is connected to a second end of the biasing element, and that the first clamping arm and the second clamping arm are maximally proximate to one another, in particular abutting against one another, in a basic position of the clip, and are movable away from one another against the action of the biasing element from the basic position into an opening position. The medical clip is formed at least partially, in particular completely, by a generative manufacturing process. The medical clip is configured having at least one first clip portion and at least one second clip portion. The at least one second clip portion is formed directly onto the at least one first clip portion by a generative manufacturing process.

### BRIEF DESCRIPTION OF THE DRAWING FIGURES

The foregoing summary and the following description may be better understood in conjunction with the drawing figures, of which:

FIG. 1 shows a schematic perspective total view of a first embodiment of a medical clip;

FIG. 2 shows a schematic perspective total view of a further embodiment of a medical clip;

FIG. 3 shows a schematic partial view of a tool for biasing a biasing element before forming on clip portions by means of a generative manufacturing process;

FIG. 4 shows a schematic view similar to FIG. 3 with clip portions partially formed onto the biasing element;

FIG. 5 shows a schematic view similar to FIG. 4 with completely formed-on clip portions;

FIG. 6 shows a schematic longitudinal sectional view of an embodiment of a transition region between two clip portions;

FIG. 6a shows a cross sectional view along line 6a-6a in FIG. 6;

FIG. 7 shows a schematic longitudinal sectional view of a further embodiment of a transition region between two clip portions;

FIG. 7a shows a cross sectional view along line 7a-7a in FIG. 7;

FIG. 8 shows a schematic longitudinal sectional view of a further embodiment of a transition region between two clip portions;

FIG. 8a shows a cross sectional view along line 8a-8a in FIG. 8;

FIG. 9 shows a schematic longitudinal sectional view of a further embodiment of a transition region between two clip portions;

FIG. 9a shows a cross sectional view along line 9a-9a in FIG. 9;

FIG. 10 shows a schematic longitudinal sectional view of a further embodiment of a transition region between two clip portions;

FIG. 10a shows a cross sectional view along line 10a-10a in FIG. 10;

FIG. 11 shows a schematic longitudinal sectional view of a further embodiment of a transition region between two clip portions;

FIG. 11a shows a cross sectional view along line 11a-11a in FIG. 11;

FIG. 12 shows a schematic perspective total view of a further embodiment of a medical clip;

FIG. 13 shows a schematic sectional view along line 13-13 in FIG. 12;

FIG. 14 shows a schematic perspective partial view of a further embodiment of a medical clip with a double web connection;

FIG. 15 shows a further schematic perspective partial view of the embodiment of a medical clip depicted in FIG. 14;

FIG. 16 shows a schematic perspective partial view of a further embodiment of a medical clip with a double web connection; and

FIG. 17 shows a further schematic perspective partial view of the embodiment of a medical clip depicted in FIG. 16.

### DETAILED DESCRIPTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a medical clip, in particular in the form of an aneurysm clip, which comprises a first clamping arm, a second clamping arm, and a biasing element, wherein the first clamping arm is connected to a first end of the biasing element, wherein the second clamping arm is connected to a second end of the biasing element, and wherein the first clamping arm and the second clamping arm are maximally proximate to one another, in particular abutting against one another, in a basic position of the clip and are movable away from one another against the action of the biasing element from the basic position into an opening position, wherein the medical clip is made partially by a generative manufacturing process, wherein the medical clip comprises at least one first clip portion and at least one second clip portion, and wherein the at least one second clip portion is formed directly onto the at least one first clip portion by a generative manufacturing process.

The proposed further development of known medical clips makes it possible, in particular, to use different manufacturing processes for producing the medical clip. For example, the biasing element, which requires, in particular, other mechanical properties than the clamping arms, may be made of another material or by another manufacturing process. Forming the clip at least partially by a generative manufacturing process also makes it possible to provide only a minimal number of parts of medical clips, for example biasing elements. Clamping arms and further parts of the clip can then be materially bonded to the provided parts by a generative manufacturing process. Thus, in particular, patient-specific clips can be provided in a simple manner. In addition, hundreds of different clips no longer have to be provided. The number of clips to be provided can thus be reduced as a result of the generative manufacturing process. Furthermore, mechanical properties of different regions of the clip can be optimized. For example, the biasing element may be made by a generative manufacturing process. The clamping arms may, in particular, be formed directly onto the biasing element by a generative process. In particular, in order to equip different regions of the clip with different optimized properties, it is advantageous for the medical clip to be configured having at least one first clip portion and at least one second clip portion. By directly forming the at least one second clip portion onto the at least one first clip portion, in particular, connecting processes for connecting the clip portions to one another can thus be foregone, which can lead to the input of heat into the clip portions in an undesired manner. In particular, a connection of the clip portions by welding can be avoided. An undefined input of heat can lead, in particular, to a thermal treatment of the biasing element, as a result of which a closing force of the biasing element and thus of the medical clip may change in an undefined manner. Directly forming on in this sense means, in particular, that the clip portions are not formed separately and then connected to one another, for example by welding or soldering, but instead that the at least one second clip portion is directly formed onto the provided at least one first clip portion. The at least one first clip portion may also be made by a generative manufacturing process. The clip portions may be made of the same material or of different materials. The at least one second clip portion may, in particular, be formed patient-specifically and adapted to a physiological situation of the patient.

The first clamping arm and/or the second clamping arm are preferably formed by a generative manufacturing process. For example, they may be materially bonded to a biasing element, which is formed either also by a generative manufacturing process or conventionally in the form of a leg spring formed by cold forming. Forming the clamping arms by a generative manufacturing process enables, in particular, an optimal adaptation to patient-specific requirements. Ultimately, a surgeon is then no longer limited to a concrete supply of medical clips, but instead is able to have a medical clip produced with clamping arms that are optimally adapted to the patient depending on a treatment situation and to apply said clip. Furthermore, generatively manufactured clamping arms have the advantage, in particular, that they are relatively soft. This makes it possible to adapt them even further in form and shape to individual requirements of a patient. When the clamping arm is brought into its final form, it can be solidified by hardening, for example in a further process step, such that it permanently retains its form.

It is favorable if the biasing element is formed by a generative manufacturing process. Clamping arms can be joined to such a biasing element, for example, in a materially bonded manner, by a generative manufacturing process. Alternatively, it is also possible to directly form a biasing element, by way of a generative manufacturing process, onto clamping arms made by a cold forming process.

In accordance with a further preferred embodiment of the invention, provision may be made that the first clamping arm comprises an end pointing toward the biasing element, that the second clamping arm comprises an end pointing toward the biasing element, that the end of the first clamping arm pointing toward the biasing element is connected to the first end of the biasing element by way of a first connecting portion, and that the end of the second clamping arm pointing toward the biasing element is connected to the second end of the biasing element by way of a second connecting portion. This design makes it possible, in particular, to set an opening width of the clip in the desired manner. In particular, it is possible to predetermine a spacing of the clamping arms in the basic position by way of the connecting portions. The connecting portions may either be formed in one piece with the biasing element by a cold forming process or be materially bonded to the biasing element by a generative manufacturing process.

It is favorable if the first connecting portion and the second connecting portion intersect in a connection region of the clip. Such a configuration of the clip makes it possible, in particular, to open the clip, i.e., to move the clamping arms form the basic position into an opening position in which they are further apart from one another than in the basic position, by compressing the biasing element, for example by twisting a leg spring. Furthermore, a guidance of the clamping arms relative to one another when opening and closing the clip can also be predetermined by the intersection of the connecting portions.

In order to improve a guidance of the clamping arms when opening and closing, it is advantageous if the connection region is configured in the form of a box lock. In particular, so-called “scissoring” can be largely avoided. The clamping arms are thus not able to slide past one another and thereby damage tissue clamped between them due to shearing.

A box lock can be achieved in a simple manner, for example, by the one of the two connecting portions being configured having a connection region perforation and by the other one of the two connecting portions passing through the connection region perforation. The clamping arms can thus be guided in a defined manner when opening and closing.

It is advantageous if the first connecting portion and/or the second connecting portion are formed by machining. In particular, a required precision and a desired surface quality in the connection region can be achieved in this way. For example, one or both connecting portions may be formed in one piece, in particular monolithically, with the biasing element. A respective clamping arm can then be formed onto the connecting portions, for example on the distal side, by a generative manufacturing process.

It is favorable if the first connecting portion and/or the second connecting portion are formed by a generative manufacturing process. If, in particular, the connecting portion with the connection region perforation is formed by a generative manufacturing process, the otherwise typical assembly effort for affixing a closure plate, for example by welding, which closure plate closes the connecting portion that is of open configuration on one side for forming the connection region perforation, is then no longer necessary. This simplifies the production of the medical clip. Here, it may optionally be favorable if one or both connecting portions that are formed by a generative manufacturing process are reworked by a machining process. Thus, in particular, a surface quality of the portions or parts of the clip that are formed by a generative manufacturing process can be improved. If one or both connecting portions are formed by a generative manufacturing process, the clamping arms can then be formed directly onto the connecting portions by a generative manufacturing process.

In accordance with a further preferred embodiment of the invention, provision may be made that the first clamping arm comprises a first clamping face, that the second clamping arm comprises a second clamping face, and that the first clamping face and the second clamping face abut against one another or substantially abut against one another in the basic position. A defined basic position for the medical clip can be predetermined in this way.

It is advantageous if the first clamping arm and the second clamping arm are biased against one another in the basic position. In this way, in particular, a defined closing force can be predetermined for the medical clip, for example by the biasing element that biases the two clamping arms against one another in the basic position.

The medical clip can be formed in a simple manner if the biasing element is configured in the form of a coil spring with at least one complete winding. However, the coil spring may also comprise two, three, or more complete windings.

In order to obtain a biasing element with high strength, it is favorable if the biasing element is formed by a cold forming process.

The biasing element can be formed from a wire in a simple manner, in particular by winding into a coil spring.

In order to avoid or at least to minimize rejection reactions upon insertion of a medical clip in a human or animal body, it is favorable if the clip is made of at least one biocompatible material. In particular, it may be made only of one single biocompatible material. If the clip is made only of one single biocompatible material, in particular, a forming of parts, for example clamping arms, onto connecting portions or a biasing element of the clip that are made of the same material, but of a solid material, can be achieved in a simple manner. Different biocompatible materials have the advantage, in particular, that different regions or portions of the clip can be selected depending on their function, in particular, in order to form a biasing element with high strength on the one hand and clamping arms that are gentle on tissue on the other hand.

The biocompatible material is preferably a metal or a plastic. The metal may be, e.g., titanium. For example, polyetheretherketone (PEEK) may be used as the plastic.

It is favorable if the at least one first clip portion is formed by a cold forming process or by a generative manufacturing process. Cold forming is advantageous, in particular, if the clip portion is to have particularly high strength properties. Such a clip portion can then be combined with a second clip portion, the form of which can be achieved in a simple manner by a generative manufacturing process. For increasing the strength of a generatively manufactured clip portion, in particular, hot isostatic pressing (HIP process) may also be used.

In accordance with a further preferred embodiment of the invention, provision may be made that a transition of the at least one first clip portion to the at least one second clip portion defines a transition region. For example, a transition region may be defined between the biasing element and a connecting portion or between a connecting portion and a clamping arm. In particular, the two clip portions that are connected to one another by the transition region, on the one hand, may be formed by a cold forming process and/or by machining and the other clip portion, on the other hand, may be formed by a generative manufacturing process. Different manufacturing processes may thus meet one another in the transition region. The transition region is, in particular, a region of connection between two clip portions, which extends over a finite length that may be very small under certain circumstances. Thus, for example, two different materials or materials of different forms may meet one another in the transition region. In particular, a material bond between two clip portions in the transition region is achieved.

It is advantageous if the transition region defines a transition region longitudinal axis and extends between a first transition region end and a second transition region end, if between the first transition region end and the second transition region end a cross section transverse to the transition region longitudinal axis is defined at least partially by the at least one first clip portion and partially by the at least one second clip portion. If, in other words, the medical clip is severed in the transition region, regions become visible in the cross section that are formed by the at least one first clip portion on the one hand and by the at least one second clip portion on the other hand. In the transition region between the two transition region ends, the clip is thus formed by the two clip portions materially bonded to one another.

It is advantageous if at the first transition region end a cross section transverse to the transition region longitudinal axis is defined completely by the at least one first clip portion, and if at the second transition region end a cross section transverse to the transition region longitudinal axis is defined completely by the at least one second clip portion. In this way, the transition region ends can be unambiguously defined, because cross sections transverse to the transition region longitudinal axis are each defined only by one of the two clip portions. A length of the transition region can thus be unambiguously defined, namely as a distance between the two transition region ends.

It is favorable if the transition region defines a contact face between the at least one first clip portion and the at least one second clip portion and if the contact face corresponds at least to a cross sectional area of the clip in the transition region transverse to the transition region longitudinal axis. The contact face may thus correspond to or be larger than the cross sectional area of the clip in the transition region. If, for example, the first clip portion ends with an end face transverse to the transition region longitudinal axis, thus is the contact face defined. In this embodiment, the second clip portion may adjoin in surface-to-surface contact against the end face of the first clip portion. An end face of the second clip portions, which faces in the direction toward the first clip portion and is then, for example, materially bonded to the first clip portion, then has the same cross section and thus the same cross sectional area as the end face of the first clip portion.

In order to improve a connection between the clip portions, it is advantageous if the contact face is at least 50% larger than a cross sectional area of the clip in the transition region transverse to the transition region longitudinal axis. In particular, the contact face may be at least 100% larger. The larger the contact face is in proportion to the cross sectional area of the clip in the transition region, the better a connection between the two clip portions is. For example, the contact face can be enlarged in a simple manner if in the transition region undercuts are formed on the first and/or second clip portion.

It is advantageous if the transition region extends over a length, which corresponds to at least about 50% of a diameter of the transition region. In particular, the length may correspond to at least about 100% of a diameter of the transition region. Providing a length of the transition region as specified enables, in particular, an improved connection between the two clip portions in the transition region. In particular, a size of the contact face can also be enlarged by increasing the length of the transition region.

It is favorable if formed on one of the clip portions is a connecting projection pointing in the direction toward the at least one other clip portion and if formed on the at least one other clip portion is a connecting projection receptacle, which accommodates the connecting projection in a force-locking and/or positive-locking and/or materially bonded manner. This configuration makes it possible, in particular, to additionally stabilize a material bond between the two clip portions. In particular when one of the clip portions is formed by a cold shaping process, a permanent and secure connection between the clip portions can thus be achieved, for example even when one of the clip portions is formed by a generative manufacturing process.

In order to further improve a connection of the clip portions, it is favorable if at least one undercut is formed on the connecting projection and/or on the connecting projection receptacle. In particular, a secure positive-locking connection between the clip portions can thus be achieved, namely independently of whether they are additionally materially bonded to one another or not.

A positive-locking connection can be formed in a simple manner if the undercut is open in a direction transverse, in particular perpendicular, relative to the transition region longitudinal axis. In particular, undercuts can thus be achieved in a simple manner, for example by machining, on a clip portion formed by a cold forming process.

A good and secure connection of the two clip portions to one another in the transition region can be achieved, in particular, by the undercut being configured in the form of a groove. In particular, the groove may be configured in the form of an annular groove, for example in the form of a peripheral annular groove. An interengagement of the clip portions on all sides in the transition region can thus be achieved. In particular, easy removal of the clip portions from one another can thus be effectively prevented.

In order to simplify the production of the medical clip, it is favorable if the connecting projection and/or the connecting projection receptacle are of rotationally symmetrical configuration.

The connecting projection preferably defines a projection end, which is of pointed or rounded configuration. A contact face between the two clip portions can thus be enlarged in a simple manner.

It is advantageous if the contact face extends at least partially, in particular completely, transversely to the transition region longitudinal axis. In particular, it may also extend in parallel to the transition region longitudinal axis. Transverse to the transition region longitudinal axis is advantageous, in particular, for implementing undercuts, for example.

It is favorable if an angle of inclination between the transition region longitudinal axis and the contact face is in a range of about 35° to about 55°. In particular, it may be about 45°. Inclining the contact face at an angle of inclination in the specified range relative to the transition region longitudinal axis makes it possible in a simple manner to enlarge the contact face and thus to improve the connection between the clip portions.

The contact face is preferably planar or curved. For example, the contact face may be convexly curved facing away from the clip portion that is not formed by a generative manufacturing process. Such an end of the clip portion can be achieved, in particular, in a simple manner by machining or cold forming.

Being able to shape certain regions of the medical clip in the desired manner is easily possible, in particular, due to the fact that the generative manufacturing process is laser deposition welding or a thermal spraying process, in particular a metal powder application process (MPA). In particular, with these processes, regions of the clip or clip portions can be shaped in the desired manner, in particular specific to a patient.

In accordance with a further preferred embodiment of the invention, provision may be made, in particular also in the case of a medical clip of the kind described at the outset, that the biasing element is configured in the form of a coil spring with at least one complete winding, and that a winding receptacle is formed on the biasing element in the region of the first end and/or the second end, and that the winding receptacle is configured pointing in the direction toward the adjacent winding. The winding receptacle increases, in particular, a distance between an outer surface of the winding receptacle and an outer surface of the winding of the coil spring located opposite the winding receptacle. Thus, when twisting the coil spring, the first end can be completely or substantially completely prevented from coming into contact with the winding of the coil spring, for example when the clip is opened. In other words, a collision of the coil spring with, in particular, the connecting portion can be prevented in the described manner by one or two winding receptacles. Friction in the region of the coil spring when opening the clip can thereby be avoided or largely avoided, which enables a more exact setting of the closing forces of the clip.

The winding receptacle can be formed in a simple manner by a flattened portion or a flat recess on the biasing element. A flat recess may, in particular, be concavely curved pointing in the direction toward the adjacent winding. Such a winding receptacle can be formed in a simple manner by machining, in particular, with a blank from which the biasing element is formed.

The clip is favorably configured in the form of an aneurysm clip. This enables the use of the clip for treating aneurysms, in particular within the skull of a patient.

Further, the invention relates to a medical clip, in particular in the form of an aneurysm clip, which comprises a first clamping arm, a second clamping arm, and a biasing element, wherein the first clamping arm is connected to a first end of the biasing element, wherein the second clamping arm is connected to a second end of the biasing element, and wherein the first clamping arm and the second clamping arm are maximally proximate to one another, in particular abutting against one another, in a basic position of the clip and are movable away from one another against the action of the biasing element from the basic position into an opening position, wherein the biasing element is configured in the form of a coil spring with at least one complete winding, and wherein a winding receptacle is formed on the biasing element in the region of the first end and/or the second end, and wherein the winding receptacle is formed pointing in the direction toward the adjacent winding.

The winding receptacle can be formed in a simple manner by a flattened portion or a flat recess on the biasing element. A flat recess may, in particular, be concavely curved pointing in the direction toward the adjacent winding. Such a winding receptacle can be formed in a simple manner by machining, in particular, with a blank from which the biasing element is formed.

Further, the invention relates to method for producing a medical clip, in particular in the form of an aneurysm clip, wherein the clip is configured having a first clamping arm, a second clamping arm, and a biasing element, such that the first clamping arm is connected to a first end of the biasing element, that the second clamping arm is connected to a second end of the biasing element, and that the first clamping arm and the second clamping arm are maximally proximate to one another, in particular abutting against one another, in a basic position of the clip, and are movable away from one another against the action of the biasing element from the basic position into an opening position, wherein the medical clip is formed at least partially, in particular completely, by a generative manufacturing process, wherein the medical clip is configured having at least one first clip portion and at least one second clip portion, and wherein the at least one second clip portion is formed directly onto the at least one first clip portion by a generative manufacturing process.

As already laid out above in detail, a wide range of variants of medical clips can thus be produced in a cost-effective manner. For example, biasing elements that define a closing force of the clip can be provided in a finite number. Clamping arms that are connected directly or indirectly to the biasing element, for example, indirectly by way of connecting portions, can then be formed patient-specifically for optimal treatment, for example of aneurysms.

Due to the fact that the at least one second clip portion is formed directly onto the at least one first clip portion by a generative manufacturing process, in particular, connecting processes for connecting the clip portions to one another can be forgone, which can lead to an undesired input of heat into the clip portions. In particular, a connection of the clip portions by welding can be avoided. Such an input of heat can lead, for example, to an undefined thermal treatment, in particular, of the biasing element, as a result of which a closing force of the biasing element and thus the medical clip may change in an unknown and undefined manner. Directly forming on in this sense means, in particular, that the clip portions are not formed separately and then connected to one another, for example by welding or soldering, but instead that the at least one second clip portion is directly formed onto the provided at least one first clip portion. For example, a clamping arm or a connecting portion may form a second clip portion in this sense. Said second clip portions can be formed patient-specifically in a simple manner using the generative manufacturing process.

A patient-specific shaping of the clip can be achieved, in particular, in a simple manner by the first clamping arm and/or the second clamping arm being formed by a generative manufacturing process.

Furthermore, it may be favorable if the biasing element is formed by a generative manufacturing process. Thus, for example, a shape of the biasing element can also be formed patient-specifically. Clamping arms can also be formed by way of a generative manufacturing process onto a biasing element formed in that way. Alternatively, clamping arms may also be formed by a cold forming process and the biasing element may be directly formed on by a generative manufacturing process.

In accordance with a further preferred embodiment of the method, provision may be made that the first clamping arm comprises an end pointing toward the biasing element, that the second clamping arm comprises an end pointing toward the biasing element, that the end of the first clamping arm pointing toward the biasing element is connected to the first end of the biasing element by way of a first connecting portion, and that the end of the second clamping arm pointing toward the biasing element is connected to the second end of the biasing element by way of a second connecting portion. Providing the connecting portions enables, in particular, a connection of clamping arms on the one hand and biasing elements on the other hand that are provided in a standardized manner. For example, both the clamping arms and the biasing element may be formed by a cold forming process. A connection can then be achieved, for example, through the connecting portions, which are materially bonded to the biasing element on the one hand and are materially bonded to a clamping arm on the other hand. The shape and length of the connecting portions can predetermine, in particular, an opening width of the medical clip. In addition, the connecting portions also enable a guidance of the clip when opening and closing.

The first connecting portion and the second connecting portion are preferably configured intersecting in a connection region of the clip. Intersecting connecting portions may, in particular, abut against one another or, with little play, prevent movement laterally relative to one another, for example when opening and closing the clip.

In particular, the scissoring described above in medical clips can be prevented in a simple manner by the connection region being configured in the form of a box lock.

It is favorable if the one of the two connecting portions is configured having a connection region perforation and if the other one of the two connecting portions is configured passing through the connection region perforation. This makes it possible, in particular, to guide the one connecting portion in the connection region perforation when opening and closing the clip. In particular, the connection region perforation may be configured in the form of an elongate hole. In particular, said elongate hole may also define stops for a maximum opening of the clip and/or for a basic position of the clip in which the clamping arms are maximally proximate to one another, in particular abutting against one another.

In particular, the connecting portions can be formed with high precision and with a desired surface quality if the first connecting portion and/or the second connecting portion are formed by machining.

The production of the medical clip can be simplified, in particular, by the first connecting portion and/or the second connecting portion being formed by a generative manufacturing process. If, for example, one of the two connecting portions comprises a connection region perforation, the latter can thus be formed by a generative manufacturing process in one working step. If, by contrast, the connection region is formed by machining, a recess is formed on the one connecting portion, which after insertion of the other connecting portion has to be closed with a closure element, for example a closure plate. Closure plates of that kind are very small and have to be fixed to the connecting portion in an additional working step, for example by welding. This manufacturing effort can be simplified by generatively forming at least the one connecting portion.

It is advantageous if a first clamping face is formed on the first clamping arm, if a second clamping face is formed on the second clamping arm, and if the first clamping face and the second clamping face are configured abutting against one another or substantially abutting against one another in the basic position. This makes it possible, in particular, to form medical clips that are closed in a basic position.

It is favorable if the first clamping arm and the second clamping arm are biased against one another in the basic position. In particular, a minimum closing force of the medical clip can be predetermined in this way.

The clip can be formed in a simple manner if the biasing element is configured in the form of a coil spring with at least one complete winding. The coil spring may, in particular, be wound clockwise or counter-clockwise. In particular, the medical clip can thus be formed compactly. In addition, it is easily detectable, for example, with imaging processes, in particular by X-ray, for example in order to determine its exact position and orientation in the body of a patient.

The biasing element is favorably formed by a cold forming process. Thus, in particular, a very high strength of the clip in the region of the biasing element can be achieved.

The biasing element can be formed from a wire in a simple manner. For example, the wire can be wound into a coil spring with one, two, or more windings.

In order to avoid rejection reactions, it is favorable if the clip is made of at least one biocompatible material, in particular only of one single biocompatible material.

The at least one first clip portion is advantageously made by a cold forming process or by a generative manufacturing process. In particular, clip portions with high strength can be achieved by way of a cold forming process. First clip portions with a desired shape, in particular specific to a patient, can be formed in a simple manner by a generative manufacturing process.

It is favorable if the at least one first clip portion is configured in the form of the biasing element and if the biasing element is held under pretension when forming on the second clip portion. Holding under pretension can take place, in particular, by twisting the biasing element, if it is formed by a coil spring. This approach has the advantage, in particular, that one or two further clip portions can be formed onto the biasing element by a generative manufacturing process, for example onto the biasing element, without interfering with one another. It can thus be achieved, in particular, that the clamping arms are biased against one another when the medical clip is in a basic position in which the clamping arms abut against one another. If the biasing element is not held under pretension when forming on second clip portions, at best a basic position of the clip can be achieved in which clamping arms thereof abut against one another, but without being biased against one another.

It is advantageous if a transition of the at least one first clip portion to the at least one second clip portion is formed, which defines a transition region. Thus, in particular, two clip portions can be formed directly on one another in the transition region by one of the clip portions being formed onto the other by a generative manufacturing process. In particular, both clip portions may be formed by a generative manufacturing process.

It is advantageous if the transition region defines a transition region longitudinal axis and is configured extending between a first transition region end and a second transition region end, if between the first transition region end and the second transition region end a cross section transverse to the transition region longitudinal axis is configured in such a way that the cross section is defined at least partially by the at least one first clip portion and the at least one second clip portion. Forming a transition region between two clip portions in the described manner increases, in particular, a stability of the connection. A size of the contact face, as was described above, is thus increased, so that a material bond over a larger area is made possible.

It is favorable if the transition region is configured in such a way that at the first transition region end a cross section transverse to the transition region longitudinal axis is defined completely by the at least one first clip portion, and if at the second transition region end a cross section transverse to the transition region longitudinal axis is defined completely by the at least one second clip portion. The transition region can be parameterized as desired in the described manner, for example by a length of the transition region, i.e., a transition region length, that corresponds to a distance between the first and the second transition region end.

It is advantageous if the transition region is configured in such a way that it defines a contact face between the at least one first clip portion and the at least one second clip portion and if the contact face corresponds at least to a cross-sectional area of the clip in the transition region transverse to the transition region longitudinal axis. The contact face may thus be equal to or larger than the cross-sectional area of the clip in the transition region transverse to the transition region longitudinal axis. The larger the contact face is, the larger the abutting surface regions of the two clip portions are. Thus, with a larger contact face, a connection, in particular a material bond, between the clip portions is improved.

The contact face is advantageously configured at least 50% larger than a cross-sectional area of the clip in the transition region transverse to the transition region longitudinal axis. In particular, the contact face may be configured at least 100% larger than the cross-sectional area of the clip in the transition region transverse to the transition region longitudinal axis. A stability of the connection of the clip portions can thus be improved in a simple manner.

It is favorable if the transition region is configured extending over a length, which corresponds to at least about 50% of a diameter of the transition region, in particular at least about 100%. Configuring a transition region with a length in the specified range can, in particular, improve a stability of the connection between the clip portions. In particular, a length of the transition region may also be used to enlarge the contact face between the clip portions.

Furthermore, it is advantageous if formed on one of the clip portions is a connecting projection pointing in the direction toward the at least one other clip portion and if formed on the at least one other clip portion is a connecting projection receptacle that accommodates the connecting projection in a force-locking and/or positive-locking manner. A particularly stable connection between the clip portions can thus be achieved. In particular, it is advantageous if the connecting projection receptacle is formed by a generative manufacturing process or production process. Thus, for example, a positive-locking recess around the projection can be formed. In particular, undercuts can thus be achieved in a simple manner.

Preferably at least one undercut is formed on the connecting projection and/or on the connecting projection receptacle. By way of an undercut, in particular, a positive engagement can be achieved, which leads to an improvement in the stability of the connection between the two clip portions.

The method can be implemented in a simple manner if the undercut is formed open in a direction transverse, in particular perpendicular, relative to the transition region longitudinal axis. For example, such an undercut can be achieved by machining on one of the two clip portions.

An undercut can be configured in the form of a groove, in particular in the form of an annular groove, in a simple and cost-effective manner.

It is advantageous if the connecting projection and/or the connecting projection receptacle are of rotationally-symmetrical configuration. The production of the medical clip can thus be simplified.

It is favorable if the contact face extends at least partially, in particular completely, transversely to the transition region longitudinal axis. Such a configuration makes it possible, in particular, to materially bond the clip portions to one another bluntly with planar end faces, for example by direct generative formation. If the contact face extends only partially, in particular in a plurality of portions, transversely to the transition region longitudinal axis, a size of the contact face can thus be easily set in the desired manner in order to be able to form a permanent and stable connection between the two clip portions.

The contact face is preferably configured in such a way that an angle of inclination between the transition region longitudinal axis and the contact face is in a range of about 35° to about 55°, in particular is about 45°. A contact face can thus be formed in a simple manner and having a desired size.

The production of the medical clip can be simplified, in particular, by the contact face being configured planarly or curvedly.

It is advantageous if the connecting projection is configured defining a projection end and if the projection end is configured to be pointed or rounded. Forming a connecting projection with such a projection end makes it possible in a simple manner to form a contact face having a desired size.

In accordance with a preferred embodiment of the invention, provision may be made that laser deposition welding or a thermal spraying process, in particular a metal powder application process (MPA), is used as the manufacturing process. For example, with such a generative manufacturing process, a clip portion can be formed onto another clip portion in a desired manner, namely independently of whether the first clip portion was also made by a generative manufacturing process or by a cold forming process.

Furthermore, it may be advantageous, in particular also in the case of a method of the kind described at the outset, if the biasing element is configured in the form of a coil spring with at least one complete winding, if a winding receptacle is formed on the biasing element in the region of the first end and/or the second end, and if the winding receptacle is configured pointing in the direction toward the adjacent winding. Friction in the region of the coil spring, which can occur upon twisting said coil spring when the first and second ends come into contact with the adjacent winding when the coil spring contracts due to twisting, can thus be significantly reduced.

The clip can be formed in a simple manner if the winding receptacle is formed by a flattened portion or a flat recess on the biasing element.

In order to be able to use the medical clip, in particular, for treating cranial aneurysms, it is favorable if the clip is configured in the form of an aneurysm clip.

Furthermore, the use of one of the methods described above for producing one of the medical clips described above is proposed.

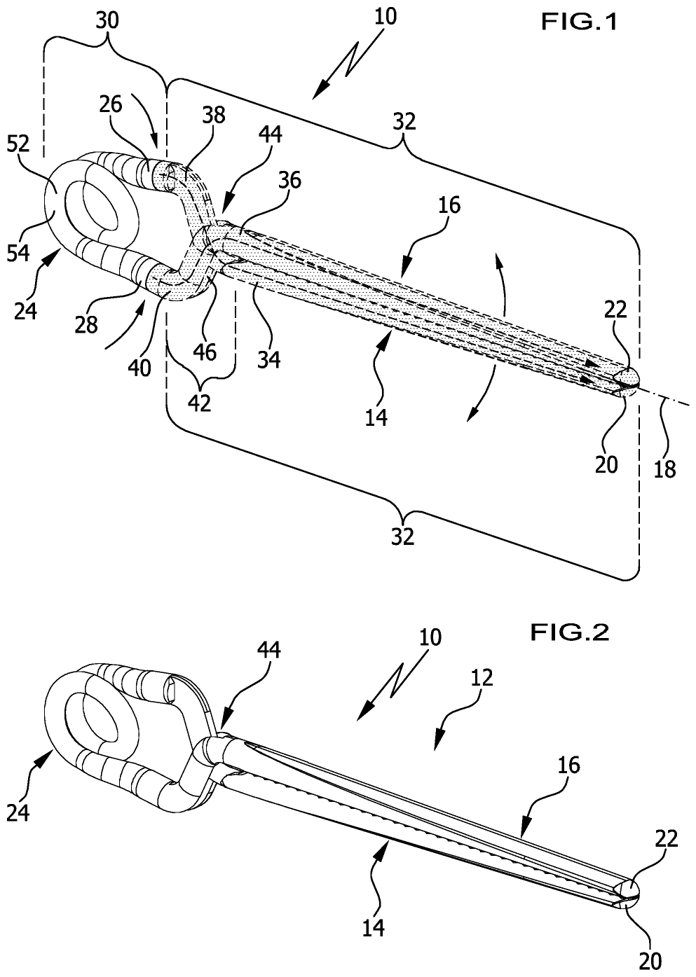

A first embodiment of a medical clip 10 is schematically depicted in FIG. 1, namely in the form of an aneurysm clip 12.

The clip 10 comprises a first clamping arm 14 and a second clamping arm 16. In the embodiment depicted in FIG. 1, these are of elongated straight configuration and, in the basic position depicted in FIG. 1, define a longitudinal axis 18 of the clip 10.

The clamping arms 14 and 16 extend on the distal side up to free ends 20 and 22.

On the proximal side, the clamping arms 14 and 16 are connected to a biasing element 24, namely the first clamping arm 14 with a first end 26 of the biasing element 24 and the second clamping arm 16 with a second end 28 of the biasing element 24.

In the basic position, the clamping arms 14 and 16 of the clip 10 are maximally proximate to one another. In the embodiment depicted in FIG. 1, the clamping arms 14 and 16 abut against one another in the basic position.

The clamping arms 14 and 16 can be moved away from one another from the basic position into an opening position. This is achieved by the ends 26 and 28 being moved toward one another. Here, the biasing element is biased or biased further and the clamping arms 14 and 16 are moved away from one another against the action of the biasing element 24 from the basic position into an opening position not depicted in the Figure.

The embodiment of the clip 10 depicted as an example in FIG. 1 comprises a first clip portion 30 and two second clip portions 32.

In the depicted embodiment, the first clip portion 30 is formed by a cold forming process. The two second clip portions 32 are formed directly onto the first clip portion by a generative manufacturing process. The clip 10 is thus formed partially by a generative manufacturing process.

The first clip portion 30 forms the biasing element 24. The second clip portions 32 are formed directly onto the ends 26 and 28 of the biasing element 24.

The first clamping arm 14 has an end 34 pointing toward the biasing element 24. Furthermore, the second clamping arm 16 has an end 36 pointing toward the biasing element 24. The end 34 is connected to the first end 26 of the biasing element 26 by way of a first connecting portion 38. The end 36 is connected to the second end 28 by way of a second connecting portion 40.

In the embodiment depicted in FIG. 1, both the clamping arms 14 and 16 as well as the two connecting portions 38 and 40 are formed by a generative manufacturing process.

The connecting portions 38 and 40 intersect in a connection region 42 of the clip 10. The connection region 42 is configured in the form of a box lock 44. Here, the first connecting portion 38 forms a male part, which passes through the second connecting portion 40 that forms a female part with a connection region perforation 46.

The first clamping arm 14 comprises a first clamping face 48. The second clamping arm 16 comprises a second clamping face 50. In the basic position of the clip 10 schematically depicted in FIG. 1, the two clamping faces 48 and 50 that face toward one another abut against one another.

In the embodiment depicted in FIG. 1, the first clamping arm 14 and the second clamping arm 16 are biased against one another in the basic position.

The biasing element 24 is configured in the form of a coil spring 52 that comprises more than one complete winding, namely approximately 1.5 windings.

The coil spring 52 is made from a wire 54 by cold forming.

The clip 10 is made of a biocompatible material. In the embodiment depicted in FIG. 1, the biocompatible material is a biocompatible metal, namely titanium. Both the first clip portion 30, which is formed by cold forming, and the second clip portions 32 are made of titanium.

Schematically depicted in FIG. 2 is a second embodiment of a medical clip, denoted as a whole with the reference numeral 10. Said clip 10 is formed completely by a generative manufacturing process.

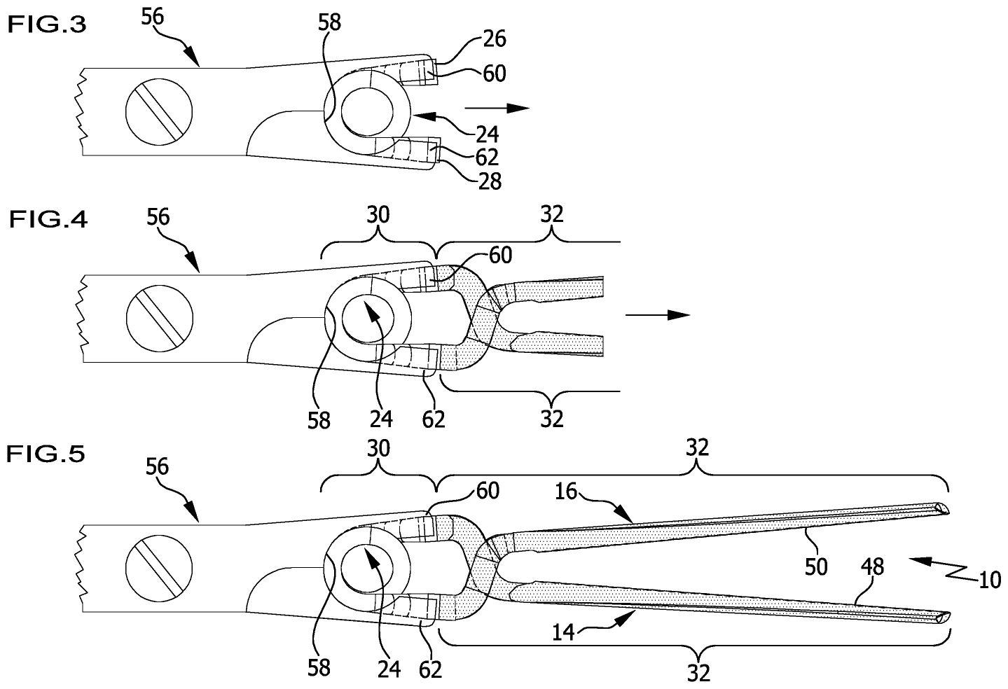

The production of the clip 10 is discussed in more detail in the following in connection with FIGS. 3 to 5.

For forming the second clip portions 32 onto the first clip portion 30, the first clip portion 30 is schematically depicted in FIG. 3 in the form of the biasing element 24, accommodated in a holding tool 56. With said holding tool 56, the ends 26 and 28 are moved somewhat toward one another, so that the biasing element 24 is held under pretension in a holding tool receptacle 58 of the holding tool 56. In this forming position schematically depicted in FIG. 3, the ends 26 and 28 point in the distal direction.

Now, as schematically depicted in FIG. 4, the second clip portions 32 can be successively directly formed onto the first clip portion 30 with a generative manufacturing process.

For forming the clip 10, laser deposition welding on the one hand and a thermal spraying process, namely a metal powder application process, may selectively be used as the generative manufacturing process.

It can easily be seen in FIGS. 4 and 5 that the clip 10 is held under pretension. When forming on the clip portions 32, the clamping faces 48 and 50 are held at a distance from one another, namely by the holding tool 56, which holds the biasing element 24 under pretension.

When the clip 10 is fully formed, as schematically depicted in FIG. 5, the biasing element 24 can be removed from the holding tool receptacle 58. For this purpose, the holding tool 56, or the two tool ends 60 and 62 thereof, which together define between them the holding tool receptacle 58, are moved somewhat away from one another, so that the clamping arms 14 and 16 can be moved toward one another until the clamping faces 48 and 50 abut against one another, as is schematically depicted in FIG. 1.

A transition region 64 is formed between the clip portions 30 and 32. Said transition region 64 is defined by a transition of the first clip portion 30 to the formed-on second clip portion 32.

The transition region 64 defines a transition region longitudinal axis 66. It extends between a first transition region end 68 and a second transition region end 70. Between the first transition region end 68 and the second transition region end 70, a cross section transverse to the transition region longitudinal axis is defined partially by the first clip portion 30 and partially by the second clip portion 32.

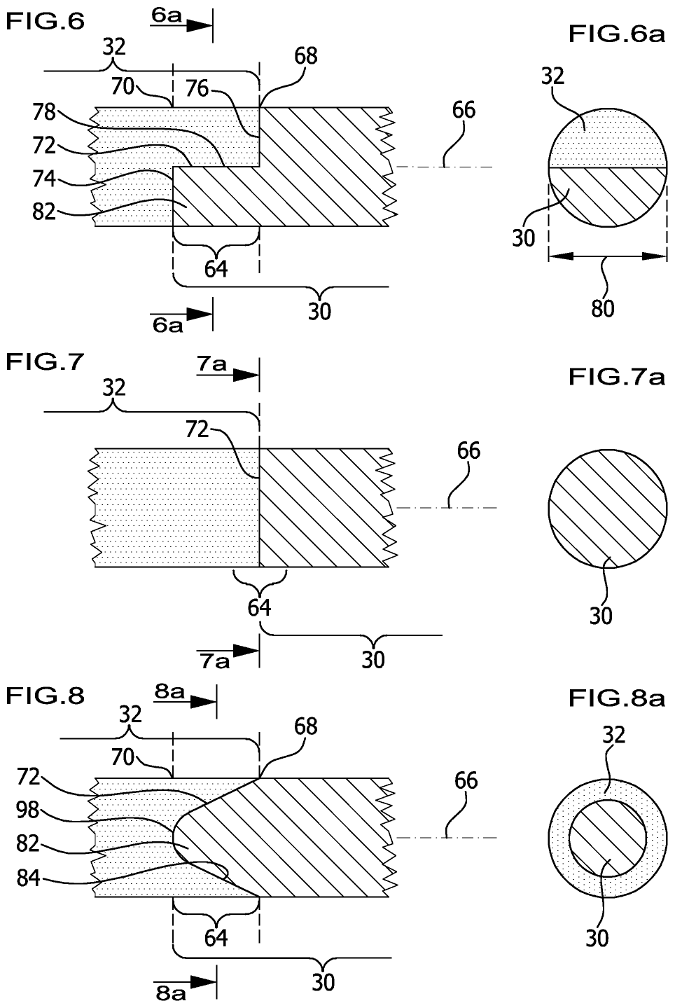

The clip 10, as it is depicted as an example in FIG. 1, has between the clip portions 30 and 32 a respective transition region 64, which is configured in the form of one of the transition regions 64 schematically depicted in FIGS. 6 to 11.

FIG. 6 shows schematically an embodiment of the transition region between two clip portions 30 and 32 in which, between the first transition region end 68 and the second transition region end 70, a cross section transverse to the transition region longitudinal axis 66 is defined half by the first clip portion 30 and half by the second clip portion 32.

FIG. 6a shows schematically a cross section between the two transition region ends 68 and 70.

The first transition region end 68 is defined such that at said end a cross section transverse to the transition region longitudinal axis 66 is defined completely by the first clip portion 30. Analogously, the second transition region end 70 is defined such that at said end a cross section transverse to the transition region longitudinal axis 66 is defined completely by the second clip portion 32.

In the transition region 64 a contact face 72 is defined between the clip portions 30 and 32. In the embodiment depicted in FIG. 6, the contact face 72 comprises three regions, namely two transverse regions 74 and 76 extending transversely to the transition region longitudinal axis 66 and a longitudinal region 78 extending in parallel to the transition region longitudinal axis 66.

A size of the contact face 72 in the embodiment depicted in FIG. 6 is greater than a cross sectional area of the clip 10 in the transition region 64 transverse to the transition region longitudinal axis 66. In the embodiment depicted in FIG. 6, the contact face 72 is enlarged by the longitudinal region 78 relative to the cross sectional area of the clip 10 in the transition region 64. The area thereof is determined as the product of the distance between the transition region ends 68 and 70 and the diameter 80 of the clip 10 in the transition region 64.

FIG. 7 shows a further embodiment of a transition region 64. Here, the clip portions 30 and 32 meet one another in surface-to-surface contact in the transition region 64. The transition region 64 is arbitrarily narrow here and, in particular, is predetermined by a roughness of the contact face 72 at ends of the clip portions 30 and 32 pointing toward one another. In this embodiment, the contact face 72 corresponds to the cross sectional area of the clip 10 in the transition region 64 transverse to the transition region longitudinal axis 66.

In this embodiment of the transition region 64 depicted in FIG. 8, a connecting projection 82 pointing in the direction toward the clip portion 32 is formed on the clip portion 30. A connecting projection receptacle 84 is formed on the clip portion 32. Said receptacle accommodates the connecting projection 82 in a force-locking and/or positive-locking and/or materially bonded manner.

In this embodiment, the connecting projection 82 is of conical configuration with a rounded end.

In this embodiment, the contact face 72 is larger than the cross sectional area of the clip 10 in the transition region 64 transverse to the transition region longitudinal axis 66.

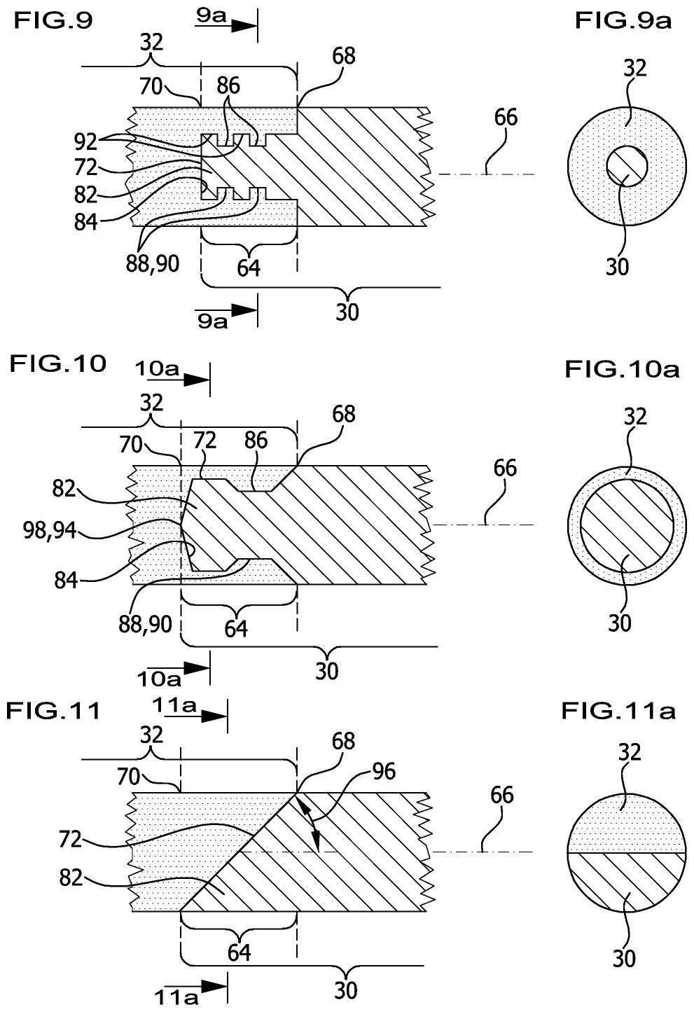

A further embodiment of a transition region 64 is schematically depicted in FIG. 9. Here, a substantially cylindrical connecting projection 82 projects from the clip portion 30 in the direction toward the clip portion 32. Formed on this connecting projection 82 are two undercuts 86, which are open in a direction transverse, namely perpendicular, relative to the transition region longitudinal axis 66.

The undercuts 68 are configured in the form of grooves 88, namely in the form of annular grooves 90.

The connecting projection 82 is accommodated in a correspondingly shaped connecting projection receptacle 84. Said connecting projection receptacle 84 comprises undercuts, namely in the form of annular grooves 92 that are open in the direction toward the transition region longitudinal axis 66.

FIG. 9a shows as an example a cross section of the transition region 64 of the embodiment depicted in FIG. 9.

A further embodiment of a transition region 64 is schematically depicted in FIG. 10. Here, too, a connecting projection 92 projects from the clip portion 30 in the direction toward the clip portion 32, said connecting projection 92 being accommodated in a correspondingly shaped connecting projection receptacle 84 in a force-locking and/or positive-locking and/or materially bonded manner.

Commencing from the first transition region end 68, the clip portion 30 tapers continuously in cross section, then remains constant on a short portion extending in parallel to the transition region longitudinal axis 66, and then again further expands continuously somewhat in the distal direction, but not in a periphery, as is defined by the first transition region end 68. On a short portion, the cross section of the connecting projection 82 remains constant in the distal direction. Following this portion, the cross section tapers continuously up to a projection end 98, which is shaped in the form of tip 94 pointing in the distal direction.

As already noted, the connecting projection receptacle 84 is configured corresponding in its form to the connecting projection 82.

The connecting projection 82 of the embodiment depicted in FIG. 10 thus also comprises an undercut 86, which is open pointing in the direction away from the transition region longitudinal axis 66. It is configured in the form of a groove 88, namely in the form of an annular groove 90.

FIG. 11 shows schematically a further embodiment of a transition region 64.

Here, the contact face 72 is inclined by an angle of inclination 96 relative to the transition region longitudinal axis 66, said angle of inclination 96 being in a range of about 35° to about 55°. In the embodiment depicted in FIG. 11, the angle of inclination 96 is 45°. In this case, the contact face 72 is of planar configuration.

In the embodiment depicted in FIG. 11, the clip portion 30 decreasing in cross section may also be referred to as a connecting projection 82. The connecting projection 82, as well as the connecting projection 82 projecting from the transverse region 76 in the embodiment of the transition region 84 depicted in FIG. 6, is not of rotationally symmetrical configuration relative to the transition region longitudinal axis 66.

By contrast, the connecting projections 82 in the embodiments of FIGS. 8, 9, and 10, as well as the corresponding connecting projection receptacles 84, are of rotationally symmetrical configuration relative to the transition region longitudinal axis 66.

In the embodiment of FIG. 7, the contact face 72 extends completely perpendicularly to the transition region longitudinal axis 66. The embodiment of FIG. 11 shows a contact face 72, which extends completely transversely to the transition region longitudinal axis 66, but at the angle of inclination 96.

The contact face 72 extends partially in parallel and partially transversely to the transition region longitudinal axis 66 in the embodiments of FIGS. 6, 8, 9, 10, and 11.

Due to the particular configuration of the transition regions 64, in particular in the embodiments of FIGS. 6, 8, 9, 10, and 11, it is possible to form the contact face 72, for example, at least 50% larger than a cross sectional area of the clip 10 in the transition region 64 transverse to the transition region longitudinal axis 66. Depending on the shape and size and number of undercuts 86, the contact face may even be more than 100% larger than the specified cross sectional area.

In the embodiments of the transition regions 64 depicted in FIGS. 6, 8, 9, 10, and 11, said transition region 64 extends over a length, which corresponds to at least about 50% of a diameter of the transition region 64.

In the embodiments of FIGS. 10 and 11, the length is even at least about 100% greater than the diameter 80 of the transition region 64.

In an embodiment not explicitly depicted, the biasing element is formed by a generative manufacturing process. Second clip portions 32 can then be formed directly onto it, also by a generative manufacturing process, as was described in more detail in connection with FIGS. 3 to 5. A clip 10 can thus be formed that is made completely by a generative manufacturing process.

In an embodiment not depicted in the Figures, the biasing element 24 is formed in one piece with the connecting portions 38 and 40, namely by a cold forming process.

In a further embodiment not depicted in the Figures, the biasing element 24 together with the connecting portions 38 and 40, which are formed in one piece by a generative manufacturing process, form a first clip portion. The clamping arms 14 and 16, which form second clip portions 32, are then directly formed onto said first clip portion by a generative manufacturing process.

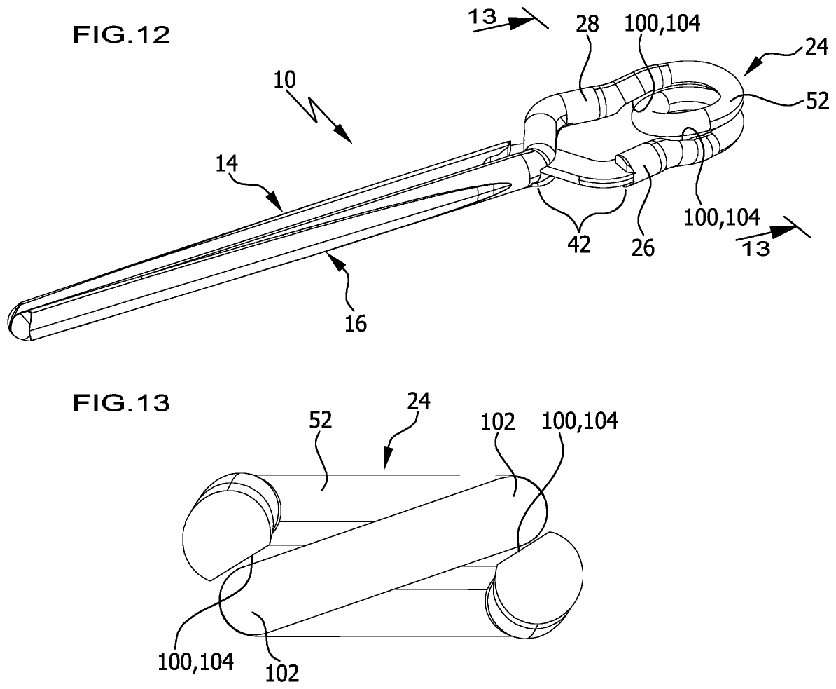

Schematically depicted in FIGS. 12 and 13 is a further embodiment of a medical clip 10. This embodiment differs from the embodiment depicted in FIG. 1, in particular, in that two winding receptacles 100 are formed on the biasing element 24, which is configured in the form of the coil spring 52. The winding receptacles 100 are formed on the biasing element 24 in the region of the first end 26 and of the second end 28. They are formed open pointing in the direction toward the adjacent winding 102.

In the embodiment depicted in FIGS. 12 and 13, the winding receptacle 100 is configured in the form of a flattened portion 104.

In an embodiment of a clip 10 that is not depicted, the winding receptacle 100 is configured in the form of a flat recess on the biasing element 24. The flat recess is concavely or slightly concavely curved pointing in the direction toward the adjacent winding 102.

The winding receptacle 100 makes it possible to twist the coil spring 52 in a substantially friction-free manner by the ends 26 and 28 being moved toward one another. When such a twisting occurs, the coil spring 52 contracts somewhat. Without the winding receptacles 100, a contact may occur here in the region in which the winding receptacles 100 are provided in the case of the embodiment of the clip 10 depicted in FIG. 12. Avoiding precisely this contact is the object of the winding receptacles 100. Thus, they make it possible to avoid possible friction as completely as possible when opening the clip 10. This makes it possible, in particular, to thoroughly test the clip 10 during and after the production thereof without it resulting in deformation of the clip 10 due to friction in the region of the biasing element 24.

This winding receptacle 100 or winding receptacles described in connection with FIGS. 12 and 13 may optionally be provided in the case of a clip 10 that is formed completely by a cold forming process.

Alternatively, one or two winding receptacles 100 may also be provided in the case of the various embodiments of medical clips that were discussed in connection with FIGS. 1 to 11.

In particular, it is possible, to provide one or two winding receptacles 100 on a biasing element 24 that is formed by a generative manufacturing process.

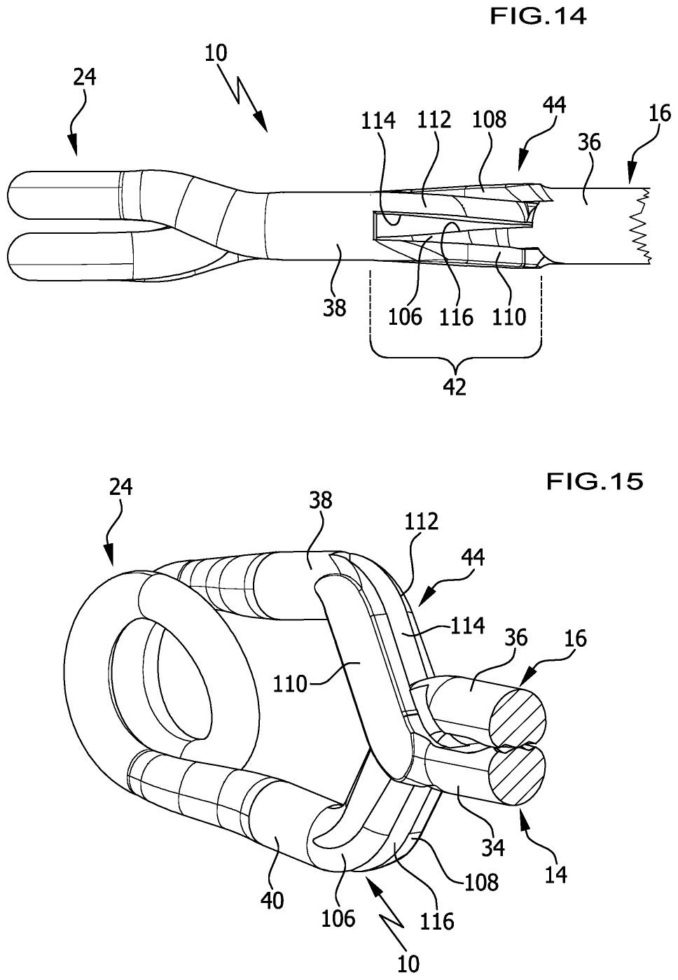

Partially depicted in FIGS. 14 and 15 is a further embodiment of a medical clip, denoted as a whole with the reference numeral 10. In this embodiment, the clamping arms 14 and 16 are not depicted in their entirety. They may be configured, in particular, as in the embodiments of medical clips 10 described above.

In the case of the medical clip 10 according to the embodiment of FIGS. 14 and 15, the design of the connection region 42 is different than in the embodiments described above. Here, the connection region 42 is configured in the form of a so-called double box lock 44. This means that not only the second connecting portion 40 is connected to the end 36 by way of two webs 106 and 108, but also the first connecting portion 38 is connected to the end 34, namely by way of two webs 110 and 112. In the other embodiments described above of FIGS. 1 to 13, only one web is provided, which connects the connecting portion 38 and the end 34 to one another. As described, said web passes through the connection region perforation 46, which is laterally delimited by two connecting webs that connect the second connecting portion 40 and the end 36 to one another.

In the case of the double box lock 44 as it is schematically depicted in FIGS. 14 and 15, the web 106 passes through a connection region perforation 114 formed between the webs 110 and 112. Furthermore, the web 112 passes through a connection region perforation 116 defined between the webs 106 and 108.

In the embodiment of the medical clip 10 of FIGS. 14 and 15, the webs 106 and 108 extend in parallel to one another, as well as the webs 110 and 112.

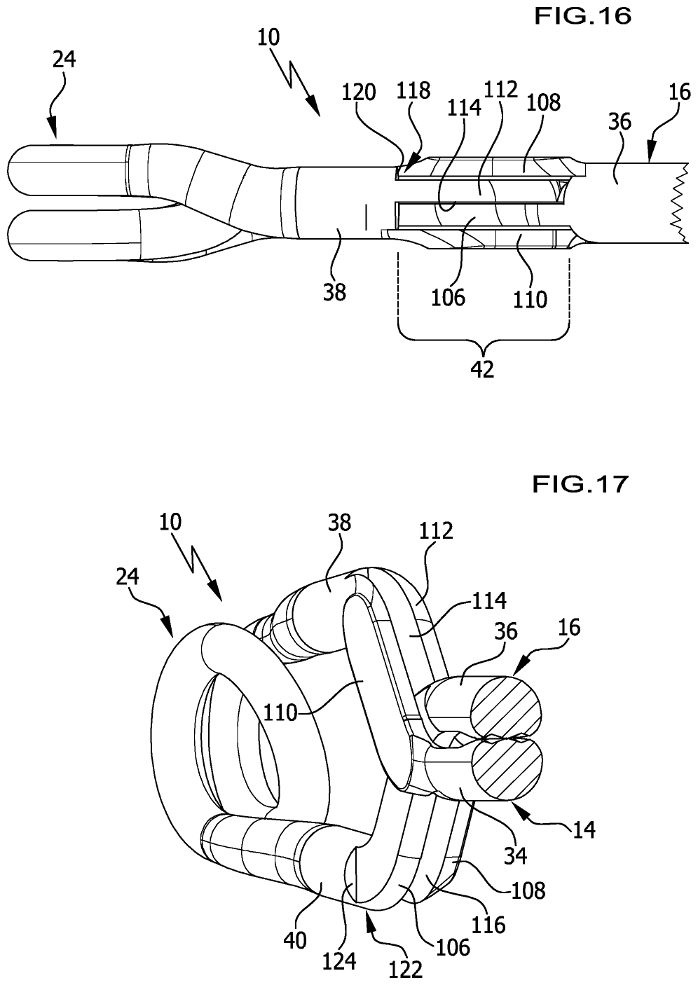

A further embodiment of a medical clip 10 is schematically depicted in FIGS. 16 and 17 in a similar manner to the embodiment of FIGS. 14 and 15. It differs in its structure from the embodiment depicted in FIGS. 14 and 15 merely in the design of a connection or the webs 106 and 108 to the connecting portion 40 and of the webs 110 and 112 to the connecting portion 38. As can be easily seen, in particular, in FIG. 16, the ends of the webs 110 and 112 connected to the connecting portion 38 are laterally offset somewhat, such that formed in the region of the web 112 is a set-back portion 118, which defines an end face 120 on the connecting portion 38 that faces in the distal direction. In an analogous manner, formed in the transition between the web 106 and the second connecting portion 40 is a further set-back portion 122, with an end face 124 facing in the distal direction.

The formation of the set-back portions 118 and 122 by laterally offsetting the respective webs 106 and 108 and 110 and 112 compared to the embodiment of FIGS. 14 and 15 creates, in particular, space for thicker webs. In the event that thicker webs are not desired, the alternative attachment of the webs 106 and 108 to the second connecting portion 40 and the attachment of the webs 110 and 112 to the first connecting portion 38 creates the option that the webs 106 and 112, which are substantially rectangular in cross section, project less far laterally beyond the connecting portions 38 and 40, which are made from a wire that is circular or substantially circular in cross section, than in the embodiment of FIGS. 14 and 15.

In particular, the set-back portions 118 and 122 prevent the connecting portions 38 and 40 from not being able to be completely accommodated in receptacles provided therefor on a clip applying instrument, which is not depicted in the Figures. Lateral protrusions, bulges, or protuberances in the regions in which the set-back portions 118 and 122 are formed may, at worst, lead to the connecting projections 38 and 40 of such medical clips, like, for example, the connecting portions 38 and 40 of the embodiment of the medical clip 10 depicted in FIGS. 14 and 15, being pushed out of receptacles provided on the clip applier instrument for the connecting portions in an undesired manner. This is prevented by the particular design of the medical clip 10 according to FIGS. 16 and 17.

Compared to the embodiment of FIGS. 14 and 15 with the obliquely extending webs 106 and 108 on the one hand and 110 and 112 on the other hand, in the embodiment depicted in FIGS. 16 and 17, as described, the webs extend obliquely only at their ends connected to the connecting portions 38 and 40 relative to a perpendicular or substantially perpendicular clip plane defined by the biasing element 24. On the other side, i.e., where the other ends of the respective webs 106 and 108 and 110 and 112 are connected to the ends 36 and 34, an offset brings no advantage, because the clip 10 abuts only with the connecting portions 38 and 40 against the clip applier instrument, but not with the clamping arms 14 and 16. Moreover, an offset of the webs 106 and 108 and 110 and 112 at the respective ends 36 and 34 would even be counterproductive, because the clamping arms 14 and 16 would then no longer lie in a plane. They would then have to additionally be brought into a parallel position by a bend or a bayonet shape.

The embodiments of FIGS. 14 to 17 are only intended to serve to explain the double box lock 44. The further components thereof may be configured, in particular, as is explained in detail above in connection with FIGS. 1 to 13.

The clips 10 described above, in particular the clips 10 described in connection with FIGS. 1 to 17, which are formed partially or completely by a generative manufacturing process, enable, in particular, a cost-effective production for a wide range of variants of medical clips 10.

The shape of the clamping arms 14 and 16 depicted in the Figures is purely exemplary. Here, instead of rectilinearly extending clamping arms 14 and 16, in principle, clamping arms 14 and 16 shaped in any way may be provided.

The production, in particular, of the second clip portions 32 opens the possibility of patient-specific production of medical clips. Furthermore, a combination of biasing elements 24 that are formed by a cold shaping process can achieve a high strength. In particular, the formation of the connection region 42 by a generative manufacturing process makes it possible to reduce the process steps for producing the clip 10.

Due to various biasing elements 24, in particular of different sizes and with different spring constants, only a small number of biasing elements of different kinds and shapes have to be provided in order to ultimately be able to form any number of different variants of medical clips. In particular, the shape and size of the clamping arms 14 and 16 can be freely predetermined and implemented as required.

By biasing the biasing element 24 when forming on the second clip portions, as described above, in particular in connection with FIGS. 3 to 5, a closing force of the clip 10 can already be set in a desired manner during the production process.

What is claimed is:

1. A medical clip comprising a first clamping arm, a second clamping arm, and a biasing element, wherein the first clamping arm is connected to a first end of the biasing element, wherein the second clamping arm is connected to a second end of the biasing element, and wherein the first clamping arm and the second clamping arm are maximally proximate to one another in a basic position of the clip and are movable away from one another against the action of the biasing element from the basic position into an opening position, wherein the medical clip is made partially by a generative manufacturing process, wherein the medical clip comprises at least one first clip portion and at least one second clip portion, and wherein the at least one second clip portion is formed directly onto the at least one first clip portion by a generative manufacturing process.

2. The medical clip according to claim 1, wherein the first clamping arm and/or the second clamping arm are formed by a generative manufacturing process.

3. The medical clip according to claim 1, wherein the biasing element is formed by a generative manufacturing process.

4. The medical clip according to claim 1, wherein the first clamping arm comprises an end pointing toward the biasing element, wherein the second clamping arm comprises an end pointing toward the biasing element, wherein the end of the first clamping arm pointing toward the biasing element is connected to the first end of the biasing element by way of a first connecting portion, and wherein the end of the second clamping arm pointing toward the biasing element is connected to the second end of the biasing element by way of a second connecting portion.

5. The medical clip according to claim 4, wherein at least one of:
a) the first connecting portion and the second connecting portion intersect in a connection region of the clip,
and
b) the one of the two connecting portions is configured having a connection region perforation and wherein the other one of the two connecting portions passes through the connection region perforation.

6. The medical clip according to claim 4, wherein the first connecting portion and/or the second connecting portion at least one of:
a) are formed by machining,
and
b) are formed by a generative manufacturing process.

7. The medical clip according to claim 1, wherein the first clamping arm comprises a first clamping face, wherein the second clamping arm comprises a second clamping face, and wherein the first clamping face and the second clamping face abut against one another or substantially abut against one another in the basic position.

8. The medical clip according to claim 1, wherein at least one of:
a) the first clamping arm and the second clamping arm are biased against one another in the basic position,
and
b) the biasing element is formed by a cold forming process.

9. The medical clip according to claim 1, wherein at least one of:
a) the clip is made of at least one biocompatible material,
and
b) the at least one first clip portion is formed by a cold forming process or by a generative manufacturing process.

10. The medical clip according to claim 1, wherein a transition of the at least one first clip portion to the at least one second clip portion defines a transition region.

11. The medical clip according to claim 10, wherein the transition region defines a transition region longitudinal axis and extends between a first transition region end and a second transition region end, wherein between the first transition region end and the second transition region end a cross section transverse to the transition region longitudinal axis is defined partially by the at least one first clip portion and partially by the at least one second clip portion.

12. The medical clip according to claim 10, wherein the transition region defines a contact face between the at least one first clip portion and the at least one second clip portion and wherein the contact face corresponds at least to a cross sectional area of the clip in the transition region transverse to the transition region longitudinal axis.

13. The medical clip according to claim 10, wherein formed on one of the clip portions is a connecting projection pointing in the direction toward the at least one other clip portion and wherein formed on the at least one other clip portion is a connecting projection receptacle, which accommodates the connecting projection in a force-locking and/or positive-locking and/or materially bonded manner.

14. The medical clip according to claim 1, wherein the biasing element is configured in the form of a coil spring with at least one complete winding, and wherein a winding receptacle is formed on the biasing element in the region of the first end and/or the second end, and wherein the winding receptacle is formed pointing in the direction toward the adjacent winding.

15. The medical clip according to claim 14, wherein the winding receptacle is formed by a flattened portion or a flat recess on the biasing element.

16. A medical clip comprising a first clamping arm, a second clamping arm, and a biasing element, wherein the first clamping arm is connected to a first end of the biasing element, wherein the second clamping arm is connected to a second end of the biasing element, and wherein the first clamping arm and the second clamping arm are maximally proximate to one another in a basic position of the clip and are movable away from one another against the action of the biasing element from the basic position into an opening position, wherein the biasing element is configured in the form of a coil spring with at least one complete winding, and wherein a winding receptacle is formed on the biasing element in the region of the first end and/or the second end, and wherein the winding receptacle is formed pointing in the direction toward the adjacent winding.

17. The medical clip according to claim 16, wherein the winding receptacle is formed by a flattened portion or a flat recess on the biasing element.

18. A method for producing a medical clip, wherein the clip is configured having a first clamping arm, a second clamping arm, and a biasing element, such that the first clamping arm is connected to a first end of the biasing element, that the second clamping arm is connected to a second end of the biasing element, and the first clamping arm and the second clamping arm are maximally proximate to one another, in a basic position of the clip, and are movable away from one another against the action of the biasing element from the basic position into an opening position, wherein the medical clip is formed partially by a generative manufacturing process, wherein the medical clip is configured having at least one first clip portion and at least one second clip portion, and wherein the at least one second clip portion is formed directly onto the at least one first clip portion by a generative manufacturing process.

19. The method according to claim 18, wherein the at least one first clip portion is formed by a cold forming process or by a generative manufacturing process.

20. The method according to claim 18, wherein the at least one first clip portion is configured in the form of the biasing element and wherein the biasing element is held under pretension when forming on the second clip portion.
